(19) United States
(12) Patent Application Publication
(10) Pub. No.: US 2014/0211160 A1
(43) Pub. Date: Jul. 31, 2014

(54) OPHTHALMOLOGIC APPARATUS AND METHOD FOR CONTROLLING THE SAME

(71) Applicant: CANON KABUSHIKI KAISHA, Tokyo (JP)

(72) Inventors: Takashi Naba, Kawasaki-shi (JP); Yukio Sakagawa, Tokyo (JP)

(73) Assignee: CANON KABUSHIKI KAISHA, Tokyo (JP)

(21) Appl. No.: 14/168,986

(22) Filed: Jan. 30, 2014

(30) Foreign Application Priority Data
Jan. 31, 2013 (JP) 2013-017663

(51) Int. Cl. A61B 3/12

(52) U.S. Cl. CPC A61B 3/12
USPC 351/206; 351/246

(57) ABSTRACT

An ophthalmologic apparatus includes an acquisition unit configured to acquire a fundus image of a subject's eye, a displacement acquisition unit configured to acquire the displacement of an imaging position between the fundus images acquired by the acquisition unit, and a display control unit configured to display on a display unit the fundus image acquired by the acquisition unit and a region of interest which is an area where a tomogram of the fundus is captured, in which the display control unit performs display control to display the fundus image and the region of interest on the display unit based on the displacement acquired by the displacement acquisition unit so that the region of interest is positioned at a predetermined position of the fundus image, and stops the display control based on the position of the subject's eye.

### BACKGROUND OF THE INVENTION

1. Field of the Invention

The present invention relates to an ophthalmologic apparatus and a method for controlling the ophthalmologic apparatus.

2. Description of the Related Art

An ophthalmic tomography imaging apparatus such as an optical coherence tomography (OCT) is capable of observing a three-dimensional state inside a retinal layer and useful for more accurately diagnosing disease. Therefore, the ophthalmic tomography imaging apparatus has drawn attention in recent years.

Japanese Patent Application Laid-Open No. 2010-227610 discusses a technique for setting an imaging parameter of an OCT tomographic image based on a measurement position specified on a fundus image of a subject's eye. Japanese Patent No. 4262603 discusses a technique for correcting the imaging position of the OCT while a fundus is being tracked to capture the OCT tomographic image since the subject's eye performs an involuntary eye movement during fixation.

The technique discussed in Japanese Patent Application Laid-Open No. 2010-227610 sets a scanner control parameter for manipulating an OCT measurement light based on the position specified on a still fundus image, however, the technique does not consider the influence of the involuntary eye movement during fixation. On the other hand, in a case where the OCT tomographic image is captured while a fundus is being tracked, as discussed in Japanese Patent No. 4262603, there is a problem in that the position of a region of interest specified on the fundus image may be different from the position where an actual tomographic image is captured.

### SUMMARY OF THE INVENTION

The present invention is directed to appropriately displaying a region of interest on a fundus image.

According to an aspect of the present invention, an ophthalmologic apparatus includes an acquisition unit configured to acquire a fundus image of a subject's eye, a displacement acquisition unit configured to acquire the displacement of an imaging position between the fundus images acquired by the acquisition unit, and a display control unit configured to display on a display unit the fundus image acquired by the acquisition unit and a region of interest which is an area where a tomogram of the fundus is captured, in which the display control unit performs display control to display the fundus image and the region of interest on the display unit based on the displacement acquired by the displacement acquisition unit so that the region of interest is positioned at a predetermined position of the fundus image, and stops the display control based on the position of the subject's eye.

Further features and aspects of the present invention will become apparent from the following detailed description of exemplary embodiments with reference to the attached drawings.

### BRIEF DESCRIPTION OF THE DRAWINGS

The accompanying drawings, which are incorporated in and constitute a part of the specification, illustrate exemplary embodiments, features, and aspects of the invention and, together with the description, serve to explain the principles of the invention.

FIG. 1 is an example illustrating a configuration of an ophthalmologic apparatus according to a first exemplary embodiment.

FIGS. 2A, 2B, and 2C are examples illustrating a configuration of an imaging unit according to the first exemplary embodiment.

FIG. 3 is a flow chart illustrating an example of processing for the ophthalmologic apparatus according to the first exemplary embodiment.

FIGS. 4A and 4B are examples illustrating a display unit of the ophthalmologic apparatus according to the first exemplary embodiment.

FIGS. 5A and 5B are charts describing the operation of a fundus tracking unit according to the first exemplary embodiment.

FIGS. 6A and 6B are examples illustrating fundus images displayed on a display unit of the ophthalmologic apparatus according to a second exemplary embodiment.

FIG. 7 is a flow chart illustrating an example of processing for the ophthalmologic apparatus according to a third exemplary embodiment.

FIGS. 8A, 8B, 8C, and 8D are examples illustrating a display unit of the ophthalmologic apparatus according to the third exemplary embodiment.

FIG. 9 is a chart illustrating a relationship between a range where an image can be captured and a region of interest.

FIG. 10 is a chart illustrating a relationship between the range where an image can be captured and the region of interest.

### DESCRIPTION OF THE EMBODIMENTS

Various exemplary embodiments, features, and aspects of the invention will be described in detail below with reference to the drawings.

A first exemplary embodiment describes an example in which the fundus is tracked when the tomographic image of a fundus is captured, and a fundus image and a region of interest indicating the position of a tomographic image are displayed based on information about tracking results so that the region of interest is positioned in a predetermined position of the fundus image.

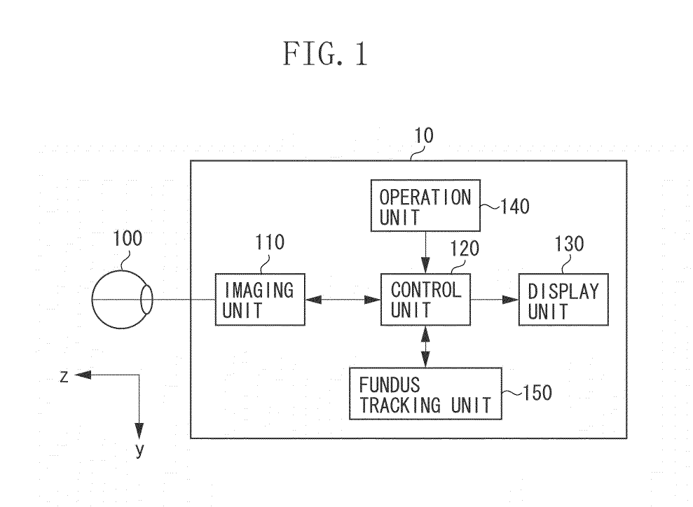

An example of a configuration of an ophthalmologic apparatus 10 according to the first exemplary embodiment is described below with reference to FIG. 1. The ophthalmologic apparatus includes an imaging unit 110, a control unit 120, a display unit 130, an operation unit 140, and a fundus tracking unit 150. The functions of the above units are described in turn below.

[Function of Imaging Unit 110]

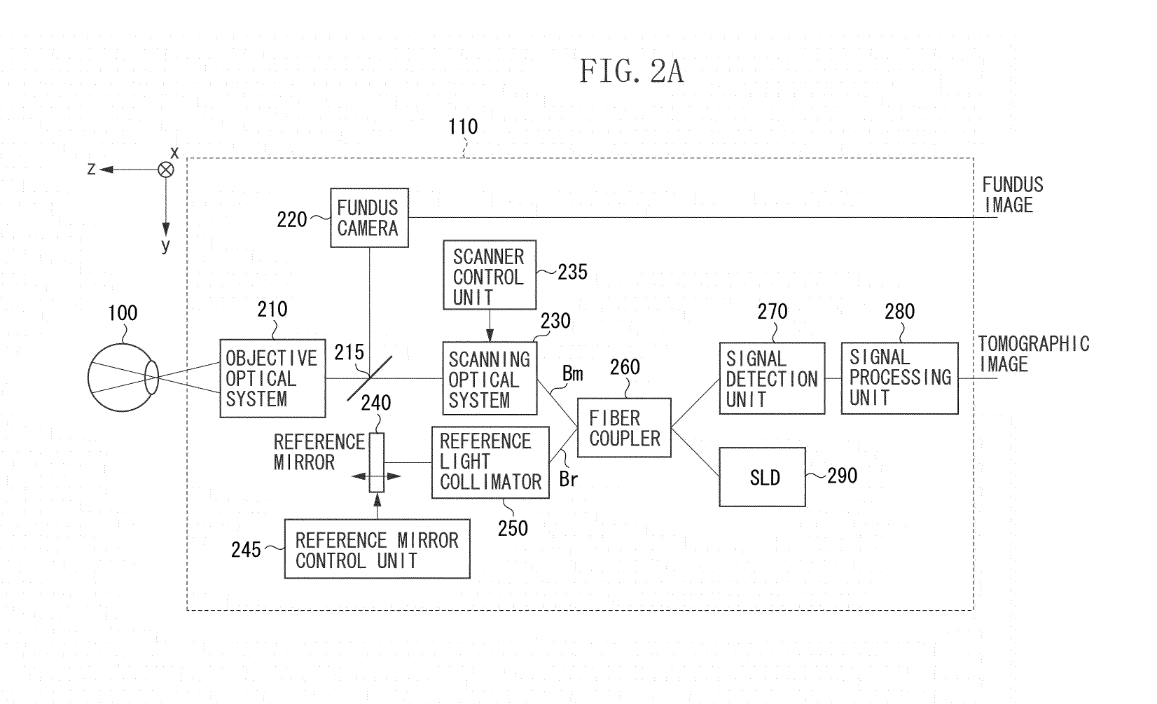

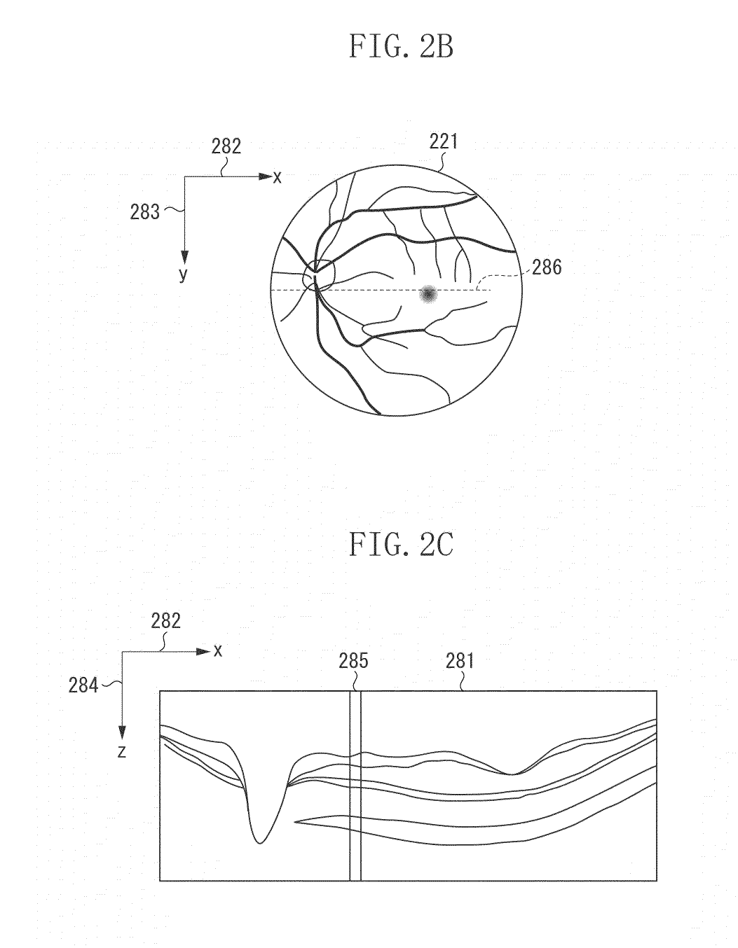

The imaging unit 110 functions as a fundus imaging unit for a two-dimensional image (a fundus image) of a subject's eye 100 or a tomographic imaging unit for the tomographic image of the subject's eye 100. An example of a configuration of the imaging unit 110 is described below with reference to FIG. 2A. The imaging unit 110 includes an objective optical system 210, a half mirror 215, a fundus camera 220, a scanning optical system 230, a scanner control unit 235, a reference mirror 240, a reference mirror control unit 245, a reference light collimator 250, a fiber coupler 260, a signal detection unit 270, a signal processing unit 280, and a super luminescent diode (SLD) 290.

The imaging unit 110 uses a spectral domain system which generates a tomographic image by Fourier-transforming a signal detected by performing spectral diffraction on interference light. In FIG. 2A, a direction perpendicular to a drawing paper surface is taken as an X axis and a measurement light scan in the X-axis direction is referred to as a horizontal scan. A downward direction with respect to the drawing paper surface is taken as a Y axis and scan in the Y-axis direction is referred to as a vertical scan.

In FIG. 2A, light emitted from the SLD 290 that is a low-coherence light source is incident on the fiber coupler 260. The fiber coupler 260 separates the incident light into a measurement light Bm and a reference light Br. The measurement light Bm is output to the scanning optical system 230 via an optical fiber. The reference light Br is output to the reference light collimator 250 via the optical fiber.

The scanning optical system 230 condenses the input measurement light Bm into a galvanomirror (not illustrated) to scan with the measurement light. The galvanomirror includes a scanner for horizontal scan and a vertical scanner for vertical scan. The scanner control unit 235 drives and controls both of the scanners. The scanning measurement light Bm reaches a retina of the subject's eye 100 via the objective optical system 210, is reflected by the retina, passes through again the objective optical system 210 and the scanning optical system 230, and reaches the fiber coupler 260. On the other hand, the reference light Br output from the fiber coupler 260 to the reference light collimator 250 is reflected by the reference mirror 240, passes through again the reference light collimator 250 and reaches the fiber coupler 260.

The measurement light Bm and the reference light Br reaching the fiber coupler 260 interfere with each other to generate an interference light. The interference light is output from the fiber coupler 260 to the signal detection unit 270. The reference mirror control unit 245 drives and controls the position of the reference mirror 240. The optical path length of the reference light is changed by changing the position of the reference mirror 240.

The detection unit 270 detects the interference light output from the fiber coupler 260 and outputs an electric interference signal to the signal processing unit 280. The signal processing unit 280 applies Fourier transformation to the interference signal to generate a signal (hereinafter referred to as “A-scan” signal) corresponding to a reflection rate along the Z direction of the retina, acquiring the tomographic image of the retina.

The fundus image is captured using the fundus camera 220 and the half mirror 215. Herein, the fundus camera 220 uses an infrared camera as an example, however, the fundus image may also be captured by a confocal scanning laser ophthalmoscope (SLO). A fixation mark is electronically generated by a fixation mark projection unit (not illustrated) and projected onto the retina of the subject's eye 100 to stabilize the fixation. The fixation mark projection unit projects the fixation mark onto the subject's eye 100 based on various parameters such as the projection position, size, shape, and a turned on/off state of light. These parameters are controlled by the control unit 120, for example. The fixation mark projection unit is an example of a fixation lamp for guiding the line-of-sight direction of the subject's eye.

Examples of the fundus image and the tomographic image acquired by the imaging unit 110 are described below with reference to FIGS. 2B and 2C. FIG. 2B and FIG. 2C illustrate a fundus image 221 and a tomographic image 281 of the retina respectively. In FIGS. 2B and 2C, an arrow 282 represents the direction of a horizontal scan (X direction), an arrow 283 represents the direction of a vertical scan (Y direction), and an arrow 284 represents the depth direction of the A-scan (Z direction).

The imaging unit 110 moves the galvanomirror of the scanning optical system 230 using the scanner control unit 235 in a main scanning direction (in this case, in a horizontal direction) to form one tomographic image 281 and causes the signal processing unit 280 to re-structure the A-scan 285 one by one. The tomographic image 281 is called a B-scan image, which corresponds to a two-dimensional cross section in a depth direction with respect to the retina and in a direction orthogonal thereto, i.e., a plane defined by X and Z axes. A dotted line 286 indicates a position where the tomographic image 281 is captured. The fundus image 221 of the subject's eye 100 is captured by the fundus camera 220.

[Function of Control Unit 120]

The control unit 120 generates imaging control information based on a signal output from the operation unit 140 which receives operation from an inspector, to transfer the information to the imaging unit 110, or cause the display unit 130 to display various images thereon. A central processing unit (CPU) executes a program stored in a memory (not illustrated) to realize the function of the control unit 120. The control unit 120 includes an imaging control unit 120A and a display control unit 120B which are not illustrated.

The imaging control unit 120A generates imaging control information according to an operation signal of an operator acquired from the operation unit 140 and outputs the imaging control information to the imaging unit 110. The imaging control unit 120A acquires the fundus image and the tomographic image of the subject's eye 100 from the imaging unit 110. The imaging control information includes information about an imaging position, imaging angle, and imaging area of the tomographic image. The imaging position, imaging angle, imaging area of the tomographic image represent the position and the area where the fundus is scanned with the measurement light to acquire the tomographic image. Those pieces of information are converted into control parameters used when the scanner control unit 235 of the imaging unit 110 controls the scanning optical system 230. Further, the imaging control information may include fixation mark control information for controlling the fixation mark for guiding the fixation of the subject's eye 100. The imaging control information may include not only the above information but also the control information of the reference mirror 240 and the focus control information of the objective optical system 210.

The display control unit 120B processes the fundus image and the tomographic image acquired by the imaging control unit 120A and causes the display unit 130 to display the processed images. More specifically, the display control unit 120B superimposes the region of interest onto the fundus image of the subject's eye 100 imaged by the imaging unit 110 to generate a synthesized fundus image according to the imaging control information generated by the imaging control unit 120A and displays the synthesized fundus image on the display unit 130. The region of interest superimposed onto the fundus image is, for example, the position where the OCT tomographic image is captured or the area where the OCT tomographic image is captured. The region of interest indicated by a line or a frame is displayed on the fundus image. Further, the region of interest indicated by a point, a circle, or a cross may be displayed on the fundus image.

The display control unit 120B also displays and controls a graphic user interface (GUI) for the operator inputting an operation. The display control unit 120B can be moved according to the instruction of the operation unit 140, for example, and displays an indicator pointing to any indication position of the display unit 130 on the display unit 130. An arrow cursor, for example, may be used as the indicator, however, the indicator is not limited to the cursor but other different indicators may be used as long as the indicators can point to any position of the display unit 130. Thereby, the change of the region of interest can be indicated.

Further, the display control unit 120B can recognize the coordinate on the display unit 130 and recognize where an indicator exists in the area of the display unit 130, based on the operation signal input from the operation unit 140. The display control unit 120B can also recognize the coordinate of the area on the display unit 130 displaying the fundus image. Accordingly, if the operation unit 140 uses a mouse, the display control unit 120B can recognize the position of the indicator on the display unit 130 moving in response to the movement of the mouse, based on an operation signal indicating the movement of the mouse. Moreover, the display control unit 120B can recognize whether the indicator moving in response to the operation of the operation unit 140 exists in an area on the display unit 130 displaying the fundus image. Still furthermore, the display control unit 120B can recognize where the indicator is displayed relative to the coordinate of the fundus image.

[Function of Display Unit 130]

The display unit 130 displays an image processed by the display control unit 120B, and a GUI layout. The display unit 130 also displays an indicator such as an arrow cursor and other various information.

[Function of Operation Unit 140]

The operation unit 140 outputs an operation signal indicating operation from the operator to the control unit 120 in response to the operation of the operator (not illustrated). Various devices such as a mouse, a keyboard, and a touch panel can be used as the operation unit 140. Assume that a mouse with a button and a wheel is used as the operation unit 140. When the mouse used as the operation unit 140 is pressed for a moment (click), the operation unit 140 outputs an operation signal indicating that the operation unit 140 is clicked, to the control unit 120. When the wheel of the operation unit 140 (the mouse) is rotated, the operation unit 140 outputs an operation signal indicating the amount of rotation of the wheel and an operation signal indicating the direction of rotation of the wheel to the control unit 120. Further, when the operation unit 140 (the mouse) is moved, the operation unit 140 outputs an operation signal indicating movement, to the control unit 120. The operation unit 140 may be composed of one device such as a mouse or a keyboard, or two or more devices. The operation unit 140 may be composed of a mouse and a keyboard, for example.

[Function of Fundus Tracking Unit 150]

The fundus tracking unit 150 calculates the amount of displacement of the fundus by analyzing the movement of the fundus of the subject's eye 100 from the fundus image captured by the imaging unit 110. In other words, the fundus tracking unit 150 is an example of a displacement acquisition unit for acquiring the displacement of an imaging position between the fundus images acquired by an acquisition unit. If there are a first and a second fundus image captured at two different times, the following processing is performed. The fundus tracking unit 150 sets a region of interest (ROI) 1 on the first fundus image and records the position of the ROI 1. The ROI 1 is a region including an image feature amount such as a strong contrast on the first fundus image. Then, the fundus tracking unit 150 searches for an ROI 2 which is the most correlated with the ROI 1 on the second fundus image. A relative difference between the positions of the ROI 1 and the ROI 2 is an amount of displacement of the fundus.

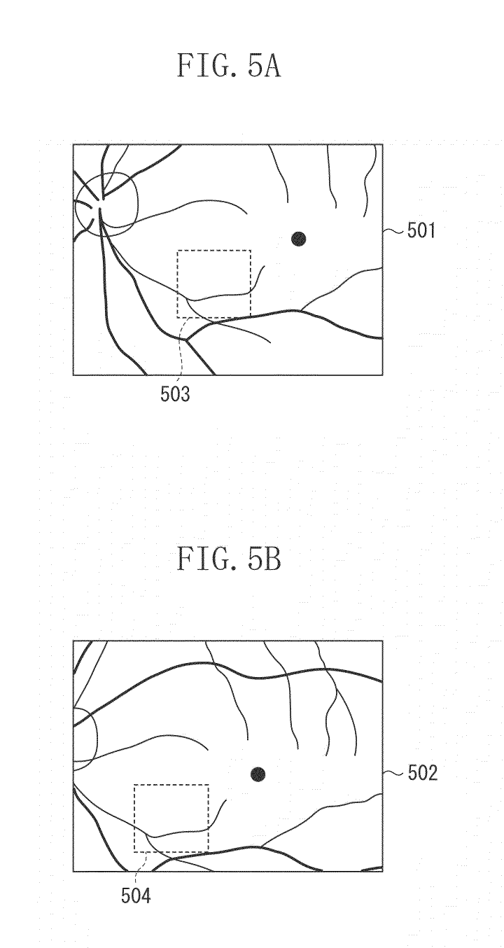

Specific examples are described below with reference to FIGS. 5A and 5B. Fundus images 501 and 502 are images of the same subject's eye 100 captured at two different times. An ROI 503 is set on a fundus image 501. As a result of search on the fundus image 502, the most correlated ROI 504 is found. If the position of the ROI 503 is (x1, y1) and the position of the ROI 504 is (x2, y2) on the coordinate system of the fundus image, the displacement of two images is represented by (x2−x1, y2−y1). The position of (x1, y1) may be any position coordinate of the ROI 503, for example, it may be the center coordinate of the ROI 503, or the coordinate of the upper left corner on the drawing paper surface, for example.

In the present exemplary embodiment, the processing using contrast or correlation is described, however, any method such as an optical flow method can be used as long as the amount of a relative displacement between images can be calculated. Two or more ROIs are set on the fundus image, for example, and a rotation amount of the fundus in addition to an amount of a parallel movement may be calculated together from the calculation results of the amount of their respective movements.

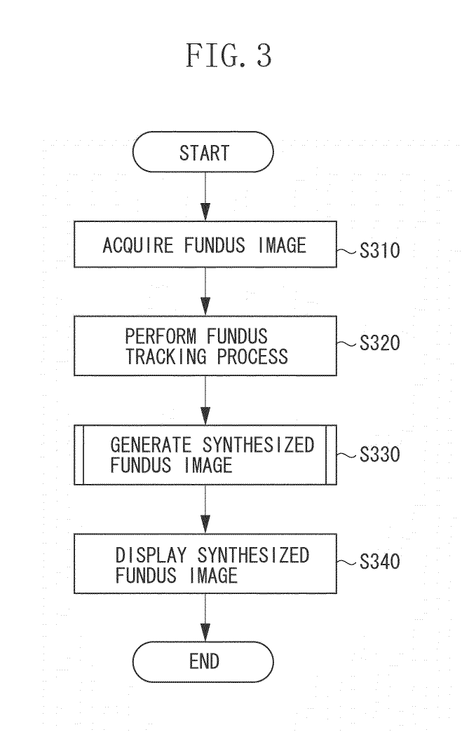

A procedure of a specific processing executed by the ophthalmologic apparatus 10 according to the first exemplary embodiment is described below with reference to a flow chart in FIG. 3.

In step S310, the imaging control unit 120A outputs a command to capture the fundus image to the imaging unit 110, to acquire the fundus image captured by the imaging unit 110. The imaging control unit 120A outputs the fundus image to the display control unit 120B and the fundus tracking unit 150. Then, the imaging control unit 120A outputs the imaging control information used for capturing the tomographic image, to the display control unit 120B.

In step S320, the fundus tracking unit 150 tracks the fundus to calculate the amount of displacement between the fundus images.

In step S330, the display control unit 120B generates a synthesized fundus image by superimposing the region of interest indicating the position where the tomographic image is captured, on the fundus image. In the present exemplary embodiment, the display control unit 120B generates the synthesized fundus image after correcting the position where a frame indicating the region of interest is superimposed on the fundus image in order to decrease the displacement, based on the amount of displacement of the fundus image calculated in step S320. As a result, the region of interest is superimposed on the same site (position) of the sequentially captured fundus image, in other words, on a specific position of the fundus image. More specifically, Assume that there are a first and a second fundus image which are captured at different times. The region of interest is superimposed on the first fundus image at a position of the coordinate (x1, y1) of the first fundus image. When the amount of displacement (dx, dy) of the first and the second fundus image is calculated, the region of interest is superimposed on the position of the coordinate (x1+dx, y1+dy) of the second fundus image. In other words, control is performed such that the position indicating the region of interest on the fundus image is moved to display the region of interest on a predetermined position.

In step S330, a sideways movement is described as an example, however, if the amount of rotation is also included in the displacement amount of the fundus image, a configuration for correcting the position of the region of interest using the amount of rotation may be adopted. The region of interest itself may be superimposed on the fundus image with the region of interest tilted. As long as the position where the region of interest is displayed can be corrected based on the displacement amount of the fundus image, any other methods may be adopted for correction.

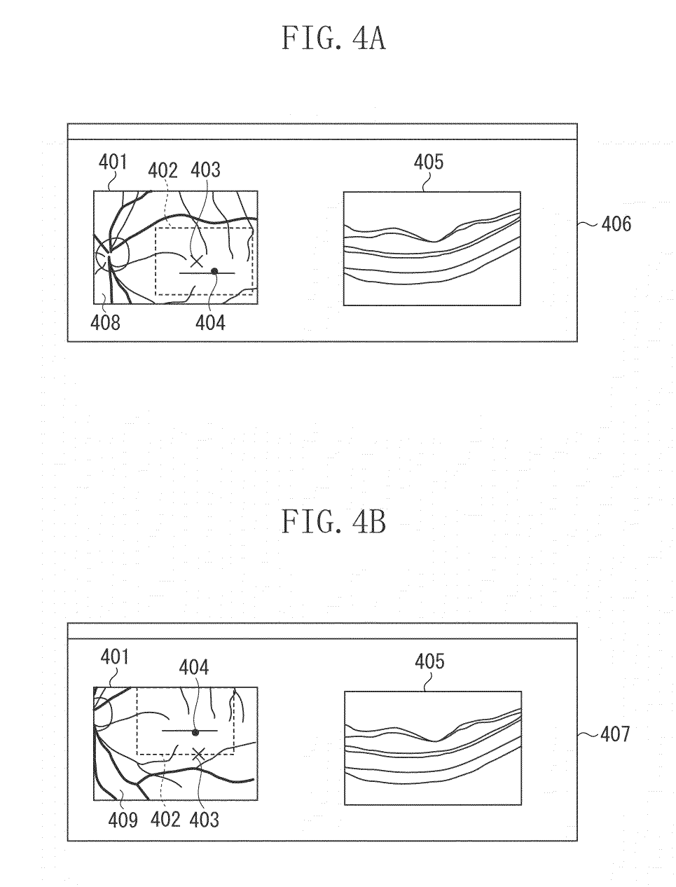

In step S340, the display unit 130 displays the synthesized fundus image generated in step S330. An example of the synthesized fundus image displayed by the display unit 130 according to the first exemplary embodiment is described below with reference to FIGS. 4A and 4B. FIGS. 4A and 4B illustrate display examples 406 and 407 as images captured at different times. A fundus image display area 401 is an area for displaying the fundus image. Fundus images 408 and 409 are displayed on the fundus image display area 401. A region of interest 402 surrounded by a dotted line indicates a region where the tomographic image is captured. A position 404 where the tomographic image indicated by a line segment is captured is one of image-capture positions in the region of interest 402. The tomographic image 405 is an image captured in the position 404 where the tomographic image is captured. As illustrated in FIGS. 4A and 4B, as a result of the subject's eye 100 performing an involuntary eye movement during fixation, the fundus images 408 and 409 are at different positions in the retina. The position on which the region of interest is superimposed is corrected on the basis of the tracking result (an amount of movement displacement) between the fundus images 408 and 409 to arrange the region of interest 402 at the same site on the fundus. The processing in the flow chart in FIG. 3 is ended here. Although the region of interest 402 is rectangular, the present invention is not limited to a rectangle, and the region of interest where the tomographic image is captured may be circular or linear. More specifically, a scanning pattern such as circle scan, cross scan, or radial scan may be taken as the region of interest 402.

As described above, according to the present exemplary embodiment, the position on which the region of interest is superimposed is corrected using tracking information between the fundus images (an amount of displacement). The region of interest indicating the area and the position where the tomographic image is captured is superimposed on the fundus image. In other words, the fundus image and the region of interest indicating the position of the tomographic image are displayed so that the region of interest is placed in a predetermined position of the fundus image. Accordingly, the region of interest can be appropriately displayed on the fundus image while the influence of the involuntary eye movement during fixation is reduced and the position where the tomographic image is captured on the fundus image can be accurately grasped.

In the present exemplary embodiment, a higher resolution of the fundus image is 600 pixels (vertical) and 800 pixels (horizontal) and a lower resolution of the fundus image is 100 pixels (vertical) and 150 pixels (horizontal). A tomographic image area is smaller than the fundus image, and a square (two-dimensional) tomographic image is composed of 1024 A-scan (horizontal) by 128 B-scan (vertical). A higher scan rate of the fundus image is 20 frames/sec and a lower scan rate thereof is 1 frame/sec.

In the first exemplary embodiment, as an example, the position where the tomographic image is captured can be accurately grasped by correcting the position where the region of interest (a region indicating the area and the position where the tomographic image is captured) is displayed on the fundus image based on tracking information between the fundus images (an amount of displacement) and by moving the region of interest to superimpose and display the region of interest. In a second exemplary embodiment, on the other hand, a method will be described in which the position where the fundus image is displayed is controlled based on the tracking information of the fundus image.

The ophthalmologic apparatus 10 of the second exemplary embodiment is similar in configuration to that of the first exemplary embodiment, so that the description of the configuration is omitted. Processing according to the second exemplary embodiment is similar to the processing illustrated by the flow chart in FIG. 3 according to the first exemplary embodiment except step S330, so that the description thereof is omitted. Step S330 executed in the second exemplary embodiment is described as step S330B.

In step S330B, the display control unit 120B generates a synthesized fundus image by superimposing the area and the position where the tomographic image is captured, on the fundus image. In the present exemplary embodiment, pixels of the fundus image are moved based on the amount of displacement between the fundus images calculated in step S320. More specifically, the amount of displacement between a first and a second fundus image captured at different times is taken as (dx, dy).

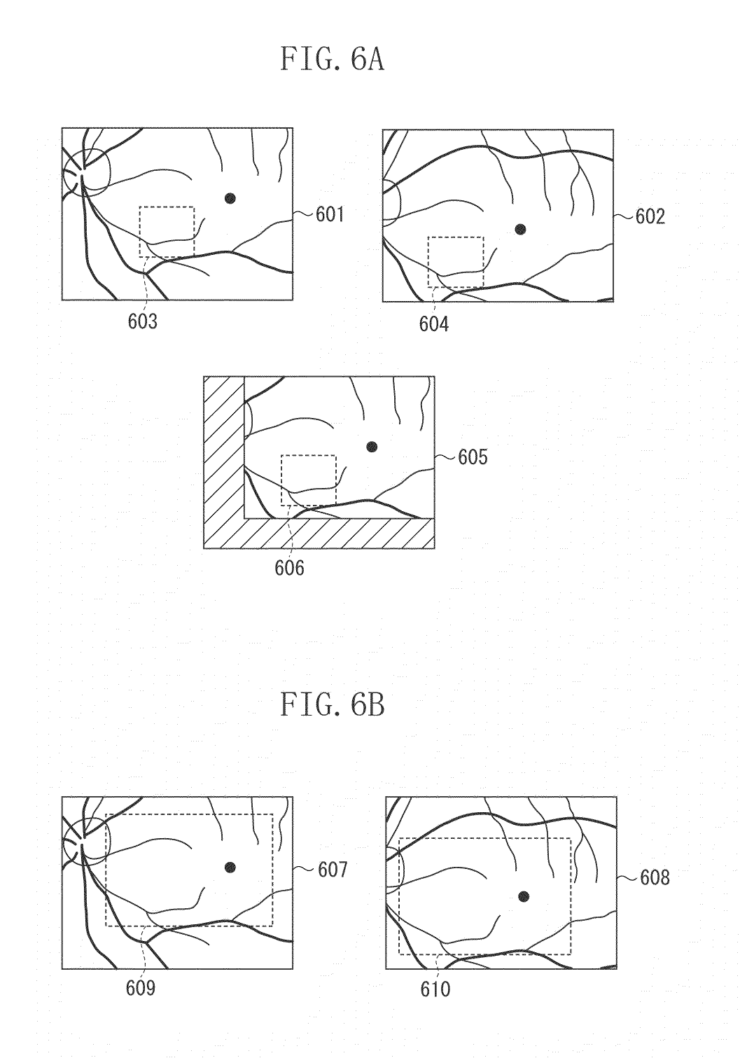

A size of the synthesized fundus image is equal to the second fundus image. The synthesized fundus image (x, y) is taken as a second fundus image (x+dx, y+dy) with respect to all pixel positions (x, y) of the synthesized fundus image. The fundus image (x, y) represents the pixel value of the pixel positions (x, y) where the fundus image exists. In other words, with reference to FIG. 6 described below, a synthesized image 605 is generated in such a way that the pixel value of a fundus image 602 is stored and the pixel value is copied at a position where an amount of displacement is cancelled.

A specific example is described below with reference to FIG. 6. Fundus images 601 and 602 are the images of the same subject's eye captured at different times. Assume that an ROI 603 is set on a fundus image 601 and an area corresponding to the ROI 603 is searched for on a fundus image 602, and as a result, an ROI 604 which is the most correlated with the ROI 603 is found. If the position of the ROI 603 is (x1, y1) and the position of the ROI 604 is (x2, y2) in the coordinate system of the fundus image, the amount of displacement between two pixels (dx, dy) is (x2−x1, y2−y1). As a result of processing in step S330B, a synthesized fundus image 605 is generated and an ROI 606 indicates the same region as the ROI 604. The fundus image on the synthesized fundus image 605 is the fundus image 602 which is moved to cancel the amount of displacement (dx, dy). A position of the fundus image 606 in the coordinate system of the synthesized fundus image 605 is equal to the ROI 603.

In such processing, an area may occur where a pixel value is unfixed such as a shaded area (a left and a lower end of the area) in the synthesized fundus image 605. These pixel values may be expressed in background color such as gray or black, for example, or may be expressed in other colors or by slanted lines. For example, the area where a pixel value is unfixed may have a pixel value at a pixel position same as the fundus image 601. Further, before the processing in step S330B is started, the copy of the first fundus image may be taken as the synthesized fundus image. The pixel value of the fundus image 602 is copied on the copy of the first fundus image based on the amount of displacement to enable eliminating of the area where the pixel value is unfixed.

The entire fundus image is moved in step S330B as an example, however, a display area of the fundus image may be clipped. The display control unit 120B may clip a part of the fundus image acquired from the imaging unit 110 and superimpose the region of interest indicating the position where the fundus image is captured, on the clipped part. Thus, the possibility that the area of the unfixed pixel value spears, is reduced.

More specifically, the first and second fundus images are captured at different times and a display area 1 to be displayed first is set on the first fundus image. The region of interest is superimposed on the image of the display area 1 and output to the display unit 130. Then, the area which is the most correlated with the display area 1 is searched for on the second fundus image and the area is taken as a display area 2. The region of interest is superimposed on the display area 2 and output to the display unit 130.

A specific example is described below with reference to FIG. 6B. Fundus images 607 and 608 are images of the same subject's eye captured at different times. A display area 609 is set on the fundus image 607 and the region of interest superimposed on the display area 609 is displayed on the display unit 130. A display area 610 which is the most correlated with the display area 609 is searched for on the fundus image 608. The region of interest is superimposed on the display area 610 and the region of interest superimposed thereon is displayed on the display unit 130. Thus, areas 609 and 610 which are parts of the fundus image are displayed on the display unit 130, so that the possibility that an area where the pixel value is unfixed appears due to the movement of a subject's eye, can be further reduced as compared with a case where all areas of the captured fundus image are displayed on the display unit 130.

As described above, according to the present exemplary embodiment, the position where the fundus image is displayed is controlled based on the tracking information about the fundus image at the time of superimposing the region of interest indicating the area and the position where the tomographic image is captured, on the fundus image. Accordingly, a less variable fundus image is captured, reducing the motion of the region of interest on the fundus image, so that the position of the area where the tomographic image is captured becomes more comprehensible.

In the first exemplary embodiment, the example is described in which the region of interest is superimposed and displayed on the fundus image to accurately grasp the position where the tomographic image is captured based on tracking information of the fundus image. In a third exemplary embodiment, a display control method for reducing the motion of the region of interest when the operator operates the region of interest is described. The ophthalmologic apparatus 10 of the third exemplary embodiment is similar to that of the first exemplary embodiment in configuration, so that the description thereof is omitted.

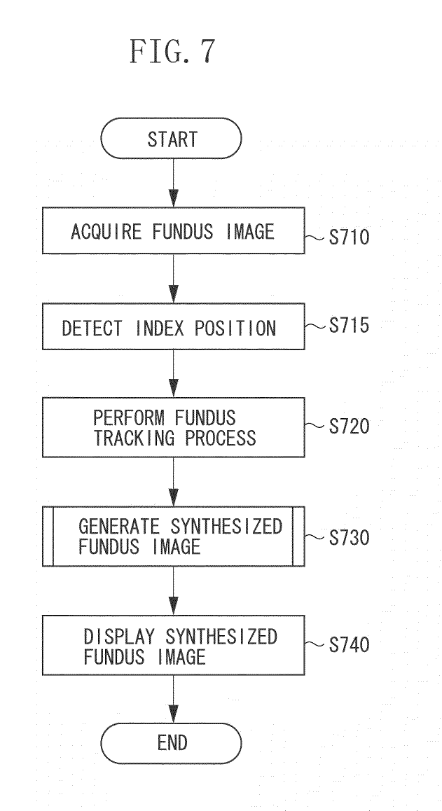

A procedure of a specific processing executed by the ophthalmologic apparatus 10 according to the third exemplary embodiment is described below with reference to a flow chart in FIG. 7. The processing in steps S710, S720, and S740 is similar to steps S310, S320, and S340 respectively, so that the description thereof is omitted.

In step S715, the operation unit 140 outputs an operation signal indicating operation from the operator to the control unit 120.

In step S730, the display control unit 120B generates a synthesized fundus image by superimposing the region of interest indicating the position where the tomographic image is captured on the fundus image. More specifically, the display control unit 120B acquires the position of an index moving on the display unit 130 according to the instruction of the operation unit 140 to determine whether the index exists on the fundus image displayed on the display unit 130. A method for synthesizing the fundus image is different according to the determination result.

Specifically, the fundus image is displayed on a part of the area of the display unit. If the index indicating any position of the display unit also exists in the area of the display unit except the part of the area, the region of interest is moved on the display unit based on the calculated displacement. On the other hand, if the index indicating any position of the display unit exists on the fundus image, the movement of the region of interest is stopped.

Thus, if the index is not found on the fundus image, the same process as that in step S330 is conducted. If the index is found on the fundus image, on the other hand, the region of interest is superimposed on the same position as the position of the region of interest of the last fundus image, instead of correcting the portion of amount of displacement of the fundus image at the time of superimposing the region of interest on the fundus image. In other words, the movement of the region of interest on the fundus image is stopped. The example described in the above makes a determination depending on whether the index is on the fundus image. However, the determination may be made depending on whether the index is in the region of interest, for example. More specifically, if the index is in the area on the display unit outside the region of interest, the region of interest is moved on the display unit based on the calculated displacement. If the index is in the region of interest, the movement of the region of interest may be stopped.

In the present exemplary embodiment, the region of interest may be operated by the operation signal input from the operation unit 140. For example, when the index is in the region of interest, a mouse click is performed using a mouse functioning as an instruction unit for changing the position where the region of interest is displayed by manipulating the position where the index is displayed. The region of interest may be moved on the fundus image by the mouse grabbing and dragging the region of interest. When the index is on the edge of the region of interest, the region of interest may be grabbed by the mouse to change the size of the region of interest. Information about the changed region of interest is transferred to the imaging control unit 120A. The imaging control unit 120A calculates a position on the fundus relative to the region of interest. The imaging control unit 120A changes imaging control information of the tomographic image of the imaging unit 110 to capture the tomographic image at a position on the fundus image and outputs the information to the control unit 110.

More specifically, if a mouse is used as the operation unit 140, for example, when the index is on the fundus image and click is performed on the fundus image, the display control unit 120B receives an operation signal according to the click. The display control unit 120B calculates a distance between the coordinate position of the index when the click is performed and a predetermined position of the area where the fundus image is displayed on the display unit 130. A unit of the distance is a pixel, for example. The position where the region of interest is displayed is changed according to the calculation result.

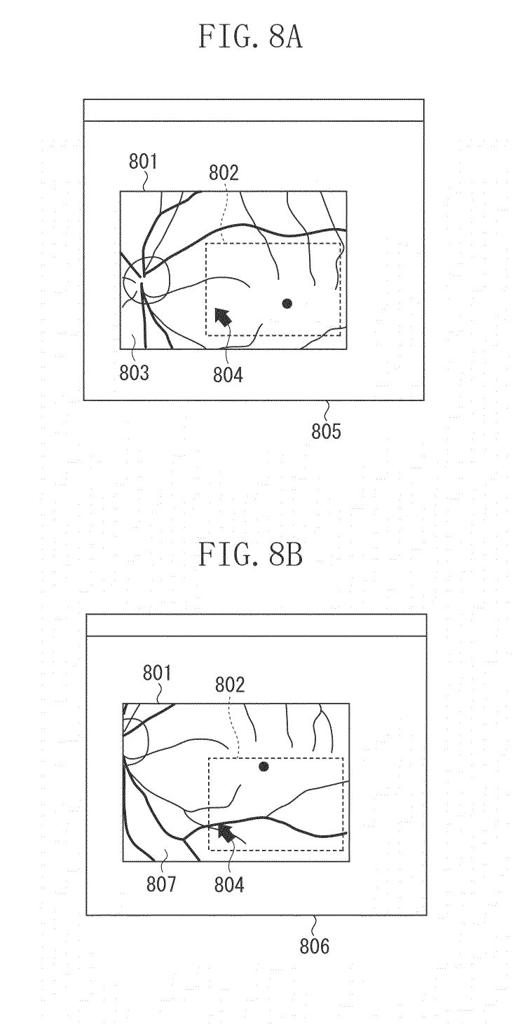

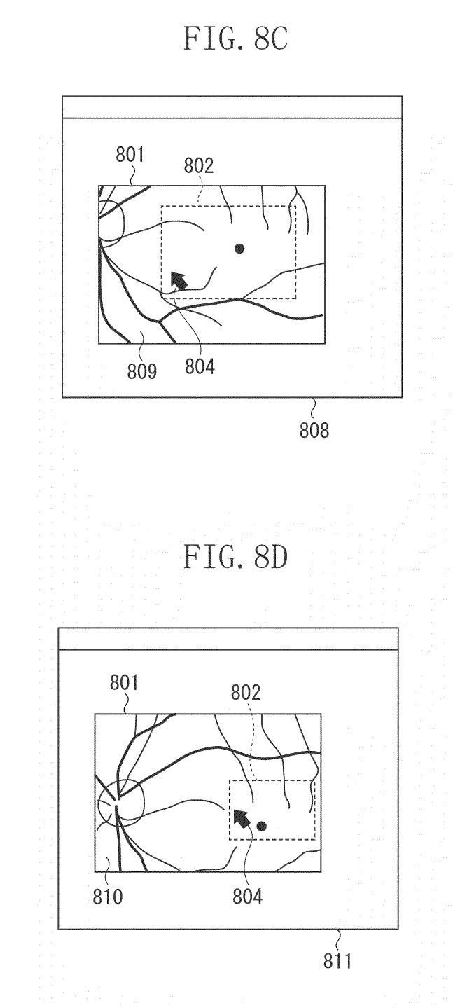

Examples of resulting display of the ophthalmologic apparatus according to the present exemplary embodiment are described below with reference to FIGS. 8A, 8B, 8C, and 8D. FIGS. 8A, 8B, 8C, and 8D illustrate display examples 805, 806, 808, and 811 at different times. A fundus image display area 801 is an area where the fundus image is displayed and fundus images 803, 807, 809, and 810 captured at different times are displayed in FIGS. 8A, 8B, 8C, and 8D. A region of interest 802 indicates a range where the tomographic image is captured. In the display example 805 illustrated in FIG. 8A, an index 804 lies in the region of interest 802, so that the position where the region of interest 802 is superimposed is the same as that in FIG. 8A as indicated by the display example 806 in FIG. 8B. When the mouse is clicked in a state of FIG. 8B, the position of the region of interest 802 is dragged by the mouse and moved (to the upper left direction on the drawing paper surface) as indicated by the display example 808 in FIG. 8C to change the position where the region of interest 802 is superimposed on the fundus image 809. FIG. 8D illustrates an example in which the size of the region of interest 802 is changed by the operation of the mouse.

As described above, according to the present exemplary embodiment, the position where the region of interest is superimposed on the fundus image is corrected based on the fundus tracking information and the index position. Accordingly it becomes easy to grasp the position where the tomographic image is captured, and manipulate the position and the range where the tomographic image is captured.

In the third exemplary embodiment, the display control method for decreasing the movement of the region of interest when the operator manipulates the region of interest has been disclosed. In a fourth exemplary embodiment, the display control of the fundus image performed when the operator manipulates the region of interest is described.

The ophthalmologic apparatus 10 of the fourth exemplary embodiment is similar in configuration to that of the first exemplary embodiment, so that the description of the configuration is omitted. Processing according to the fourth exemplary embodiment is similar to the processing illustrated by the flow chart in FIG. 7 according to the third exemplary embodiment except step S730, so that the description thereof is omitted. Step S730 executed in the fourth exemplary embodiment is described as step S730B.

In step S730B, the display control unit 120B generates a synthesized fundus image by superimposing the area and the position where the tomographic image is captured on the fundus image. More specifically, the display control unit 120B acquires the position of an index moving on the display unit 130 according to the instruction of the operation unit 140 to determine whether the index exists on the fundus image displayed on the display unit 130. A method for synthesizing the fundus image is different according to the determination result.

If the index is not on the fundus image, the same process as that in step S330 is conducted. If the index is on the fundus image, on the other hand, the same process as that in step S330B is conducted, in which the region of interest is superimposed on the same position as the position of the region of interest of the last fundus image without consideration of the amount of displacement of the fundus image, at the time of superimposing the region of interest on the fundus image. In other words, the movement of the region of interest on the fundus image is stopped. Although the example described in the above makes a determination depending on whether the index is on the fundus image, the determination may be made depending on whether the index is in the region of interest, for example. As is the case with the third exemplary embodiment, also in the present exemplary embodiment, the region of interest may be manipulated by the operation signal input from the operation unit 140.

As described above, according to the present exemplary embodiment, the method for displaying the fundus image is corrected based on the fundus tracking information and the index position. Accordingly, it becomes easy to grasp the position where the tomographic image is captured, and manipulate the position and the range where the tomographic image is captured. The position where the tomographic image is captured is less frequently changed on the fundus image even in the manipulation of the region of interest, so that the position where the tomographic image is captured on the fundus image can be more accurately specified.

In the first exemplary embodiment, no matter how much the fundus image is moved in tracking in step S320, the region of interest is moved in step S330. However, a range where the tomographic image can be captured is generally limited due to optical constraint of a lens, so that an excessively large amount of movement sometimes causes deviation from the range where the tomographic image can be captured. In the a fifth exemplary embodiment, if because of the movement of the subject's eye, the region of interest where the tomographic image is captured deviates from the range where the tomographic image can be captured, the region of interest is not moved to prevent an unclear tomographic image from being captured.

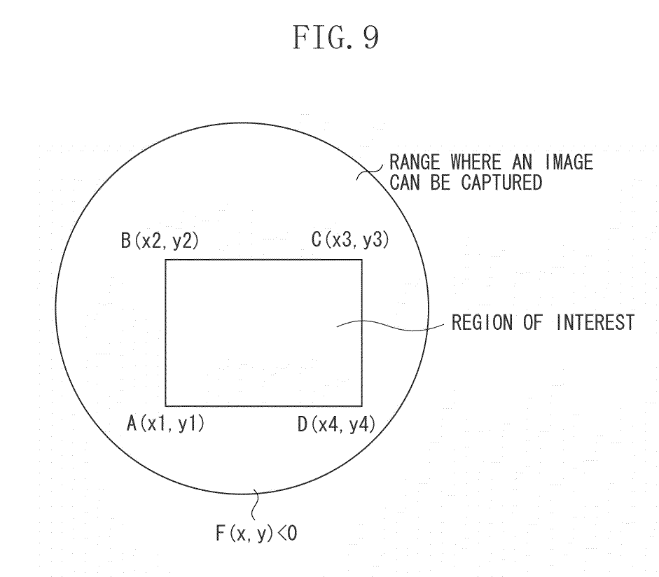

FIG. 9 illustrates a coordinate relationship between a range where an image can be captured and the region of interest. In FIG. 9, the region of interest is a square, and the coordinates of vertexes A, B, C, and D are (x1, y1), (x2, y2), (x3, y3), and (x4, y4) respectively. An evaluation function in the range where an image can be captured is f(x,y)<0. The range where an image can be captured is a circle, however, the range where an image can be captured is not limited to the circle but may take other forms.

If the range where an image can be captured is a circle with a radius a, the evaluation function is x²+y²−a²<0. If the region of interest is a rectangle, x1=x2, y1=y4, x3=x4, y2=y3.

If an image displacement is (dx, dy) in the processing of step S320, the vertexes of a new region of interest are (x1−dx, y1−dy), (x2−dx, y2−dy), (x3−dx, y3−dy), and (x4−dx, y4−dy). When the vertexes satisfy an evaluation function, the processing of step S330 is executed using the acquired amount of displacement. The evaluation function is calculated by the control unit 120, for example. If the vertexes do not satisfy the evaluation function, the display control unit 120B ends the processing without moving the region of interest. More specifically, the display control unit 120B performs display control to display the fundus image and the region of interest on the display unit based on the displacement acquired by the displacement acquisition unit so that the region of interest is positioned at a predetermined position of the fundus image. On the other hand, the display control unit 120B stops the display control according to the position of the subject's eye. In other words, the display control unit 120B performs the display control to display the fundus image and the region of interest on the display unit based on the displacement acquired by the displacement acquisition unit so that the region of interest is positioned at the predetermined position of the fundus image. On the other hand, the display control unit 120B stops the display control on the basis of the area where a tomogram can be captured. More specifically, the display control unit 120B stops moving the region of interest if the region of interest is positioned outside the area where the tomogram can be captured due to the movement of the subject's eye.

The control unit 120 calculates new dx and dy to obtain the point of the evaluation function in the vicinity of the vertexes which do not satisfy the evaluation function. In other words, the control unit 120 calculates how much the region of interest should be moved in order to include the region of interest in the area where the tomogram can be captured. The control unit 120 causes the display unit to display the region of interest in the area where the tomogram can be captured in step S330 based on the movement amount of the region of interest for including the region of interest in the area where the tomogram can be captured. Thus, the tomographic image can be surely acquired because the region of interest is included in the range where the tomogram can be captured although the area of interest is different from the original range.

In the above description, the present exemplary embodiment is applied to the first exemplary embodiment, however, the present exemplary embodiment is applicable also to the second exemplary embodiment. More specifically, if the area on the fundus included in the region of interest is positioned outside the range where an image can be captured, due to the movement of the subject's eye (if the region of interest is positioned outside the area where the tomographic image can be captured), the processing for cancelling the amount of displacement of the fundus is stopped as in the second exemplary embodiment. In other words, the display control unit 120B causes the display unit to display the fundus image without performing the processing for reducing the displacement if the region of interest is positioned outside the area where the tomographic image can be captured due to the movement of the subject's eye.

If the vertexes do not satisfy the evaluation function, irrespective whether the above processing is performed, the inspector can be notified of a deviation from the range where an image can be captured, by changing a display form such as changing a display color of the region of interest and/or blinking the region of interest. In other words, the display control unit 120B changes the display form of the region of interest if the region of interest is positioned outside the area where the tomographic image can be captured.

In the fifth exemplary embodiment, tracking is stopped if the region of interest is positioned outside the range where an image can be captured. In a sixth exemplary embodiment, the fixation lamp is moved to positively move the line-of-sight of the subject's eye, moving the region of interest to the inside of the range where an image can be captured.

In the fifth exemplary embodiment, if an image displacement is (dx, dy) in the processing of step S320 and a new region of interest is outside the range where an image can be captured, the control unit 120 moves the position of the fixation lamp by −dx in the X direction and −dy in the Y direction. In other words, if the region of interest is positioned outside the range where the tomogram can be captured, the control unit 120 changes a position where the light of the fixation lamp is projected on the subject's eye, as an example of a fixation change means. The position where the light of the fixation lamp is projected is moved to move the line-of-sight of the subject's eye and return the region of interest to the original position. The amount of change in a position where the fixation lamp is displayed does not need to be equivalent to the displacement amount of an image. The amount of change in a position where the fixation lamp is displayed may be determined based on the amount of the region of interest protruded from the range where an image can be captured, for example.

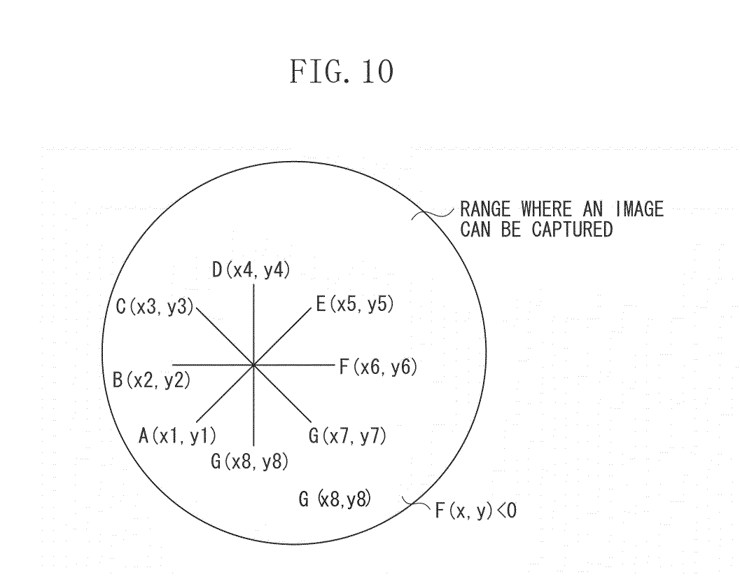

A seventh exemplary embodiment is described below. In the first exemplary embodiment, the region of interest is presumed to be a square. The region of interest can be controlled even in other forms. FIG. 10 illustrates an example of a scan pattern referred to as radial scan. Generally, in this scan pattern, scanning of a plurality of B scans is radially performed centering on a macula. In this case, the vertexes of the pattern are A, B, C, D, E, F, G, and H as illustrated in FIG. 10. It is determined in the processing of step S320 whether each point exists outside the range where an image can be captured and the processing similar to the fifth exemplary embodiment is performed to acquire the effect similar to the above exemplary embodiment with the radical scan. The scan pattern is not limited to the radical scan and a raster scan but may use a circle scan in a circular area or a cross scan in a cross area.

The present invention is not limited to the above exemplary embodiments. It is to be understood that various modifications and changes may be made without departing from the gist of the present invention. For example, the above exemplary embodiments may be combined.

### OTHER EMBODIMENTS

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™, a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-017663 filed Jan. 31, 2013, which is hereby incorporated by reference herein in its entirety.

What is claimed is:

1. An ophthalmologic apparatus comprising:
an acquisition unit configured to acquire a fundus image of a subject's eye;
a displacement acquisition unit configured to acquire the displacement of an imaging position between the fundus images acquired by the acquisition unit; and
a display control unit configured to display on a display unit the fundus image acquired by the acquisition unit and a region of interest which is a range where a tomogram of the fundus is captured;
wherein the display control unit performs display control to display the fundus image and the region of interest on the display unit based on the displacement acquired by the displacement acquisition unit so that the region of interest is positioned at a predetermined position of the fundus image and stops the display control based on the position of the subject's eye.

2. The ophthalmologic apparatus according to claim 1, wherein the display control unit moves the region of interest on the display unit based on the displacement acquired by the displacement acquisition unit to display the region of interest at the predetermined position on the fundus image.

3. The ophthalmologic apparatus according to claim 2, wherein the display control unit stops moving the region of interest if the movement of the subject's eye causes the region of interest to be positioned outside the area where the tomogram can be captured.

4. The ophthalmologic apparatus according to claim 1, wherein the display control unit displays the fundus image on the display unit in such a way as to reduce the displacement based on the displacement acquired by the displacement acquisition unit, so as to display the region of interest at the predetermined position on the fundus image.

5. The ophthalmologic apparatus according to claim 4, wherein the display control unit displays the fundus image on the display unit without performing the processing for reducing the displacement if the movement of the subject's eye causes the region of interest to be positioned outside the area where the tomogram can be captured.

6. The ophthalmologic apparatus according to claim 2, further comprising an operation unit configured to receive operation from an inspector,
wherein the display control unit displays the fundus image in a part of the area of the display unit, indicates any position of the display unit in an area on the display unit other than the part of the area, and moves the region of interest on the display unit based on the displacement acquired by the displacement acquisition unit if there is an index movable according to the operation of the operation unit, but stops moving the region of interest if there is the index on the fundus image.

7. The ophthalmologic apparatus according to claim 6, wherein the display control unit moves the region of interest on the display unit based on the displacement acquired by the displacement acquisition unit if there is the index in an area on the display unit other than the region of interest but stops moving the region of interest if there is the index in the region of interest.

8. The ophthalmologic apparatus according to claim 2, further comprising an operation unit configured to receive operation from an inspector,
wherein the display control unit displays the fundus image in a part of the area of the display unit, indicates any position of the display unit in an area on the display unit other than the part of the area, displays the region of interest at the predetermined position on the fundus image by moving the region of interest on the display unit based on the displacement acquired by the displacement acquisition unit if there is an index movable according to the operation of the operation unit, and displays the fundus image on the display unit in such a way as to reduce the displacement based on the displacement acquired by the displacement acquisition unit if there is the index on the fundus image.

9. The ophthalmologic apparatus according to claim 8, wherein the display control unit displays the region of interest at the predetermined position on the fundus image by moving the region of interest on the display unit based on the displacement acquired by the displacement acquisition unit if there is the index in an area on the display unit other than the region of interest, and displays the fundus image on the display unit in such a way as to reduce the displacement based on the displacement acquired by the displacement acquisition unit if there is the index in the region of interest.

10. The ophthalmologic apparatus according to claim 6, wherein the position and size of the region of interest can be changed according to the operation of the operation unit.

11. The ophthalmologic apparatus according to claim 6, wherein the operation unit is a mouse.

12. The ophthalmologic apparatus according to claim 2, further comprising:
a fixation lamp for guiding the line-of-sight direction of the subject's eye; and
a fixation change unit configured to change a position where the light of the fixation lamp is projected on the subject's eye if the region of interest is positioned outside the area where the tomogram can be captured.

13. An ophthalmologic apparatus comprising:
an acquisition unit configured to acquire a fundus image of a subject's eye;
a displacement acquisition unit configured to acquire the displacement of an imaging position between the fundus images acquired by the acquisition unit; and
a display control unit configured to display on a display unit the fundus image acquired by the acquisition unit and a region of interest which is a radial area where a tomogram of the fundus is captured;
wherein the display control unit performs display control to display the fundus image and the region of interest on the display unit based on the displacement acquired by the displacement acquisition unit so that the region of interest can be positioned in a predetermined position of the fundus image.

14. An ophthalmologic apparatus comprising:
an acquisition unit configured to acquire a fundus image of a subject's eye;
a displacement acquisition unit configured to acquire the displacement of an imaging position between the fundus images acquired by the acquisition unit; and
a display control unit configured to display on a display unit the fundus image acquired by the acquisition unit and a region of interest which is a circular area where a tomogram of the fundus is captured;
wherein the display control unit performs display control to display the fundus image and the region of interest on the display unit based on the displacement acquired by the displacement acquisition unit so that the region of interest can be positioned in a predetermined position of the fundus image.

15. An ophthalmologic apparatus comprising:
an acquisition unit configured to acquire a fundus image of a subject's eye;
a displacement acquisition unit configured to acquire the displacement of an imaging position between the fundus images acquired by the acquisition unit; and
a display control unit configured to display on a display unit the fundus image acquired by the acquisition unit and a region of interest which is an area where a tomogram of the fundus is captured;
wherein the display control unit moves the region of interest on the display unit based on the displacement acquired by the displacement acquisition unit so that the region of interest can be positioned in a predetermined position of the fundus image, and changes a display form for the region of interest if the region of interest is positioned outside the area where the tomogram can be captured.

16. A method for controlling an ophthalmologic apparatus including an acquisition unit, a displacement acquisition unit, and a display control unit, the method comprising:
acquiring a fundus image of a subject's eye with the acquisition unit;
acquiring the displacement of an imaging position between the fundus images acquired with the displacement acquisition unit; and
controlling display of the acquired fundus image and a region of interest which is an area where a tomogram of the fundus is captured on a display unit, with the display control unit;
wherein in carrying out the display control, the display control unit performs control to display the fundus image and the region of interest on the display unit based on the acquired displacement so that the region of interest is positioned at a predetermined position of the fundus image, and stops the display control based on the position of the subject's eye.

17. A non-transitory storage medium storing a program for causing a computer to execute each step of a method for controlling an ophthalmologic apparatus according to claim 16.
